(19) United States
(12) Patent Application Publication
(10) Pub. No.: US 2025/0186096 A1
(43) Pub. Date: Jun. 12, 2025

(54) SURGICAL SYSTEMS AND METHODS

(71) Applicant: INNOVASIS, INC., Salt Lake City, UT (US)

(72) Inventors: Mike MINDALA, Strongsville, OH (US); Ron GROBERG, Syracuse, UT (US); Brent FELIX, Salt Lake City, UT (US)

(21) Appl. No.: 18/950,103

(22) Filed: Nov. 17, 2024

Related U.S. Application Data
(60) Provisional application No. 63/607,178, filed on Dec. 7, 2023.

(51) Int. Cl. A61B 17/88; A61B 17/00; A61B 34/20; A61B 34/30

(52) U.S. Cl. CPC A61B 17/8875; A61B 34/30; A61B 2017/00367; A61B 2034/2072

(57) ABSTRACT

A surgical system for placement of a surgical screw into a bone may include the surgical screw having a screw head having a screw head diameter, a first and second arm, and an inside thread, and a screw body having a drive feature and a threaded portion configured to engage the bone. The system may further include a driver having a distal end to engage the surgical screw, a proximal end, an outer shaft having a threaded portion to engage the inside thread, an inner shaft having a drive tip to engage the drive feature, and a sleeve to be received within an end effector of a robotic surgical device, the sleeve having an outside and inside diameter. The outside diameter may be greater than the screw head diameter, the inside diameter may be less than the screw head diameter, and the sleeve may receive the first and second arms.

### CROSS-REFERENCE TO RELATED APPLICATIONS

The present disclosure claims the benefit of U.S. Provisional Patent Application Ser. No. 63/607,178, filed on Dec. 7, 2023 and entitled SURGICAL SYSTEMS AND METHODS, which is incorporated by reference as though set forth herein in its entirety.

### TECHNICAL FIELD

The present disclosure relates to systems and methods for use in orthopedic surgery. More specifically, the present disclosure relates to robotic surgical systems and methods for use in spinal surgery.

### BACKGROUND

Robotic-assisted surgery has expanded the capabilities of modern medicine by offering enhanced precision, dexterity, and control to surgeons during surgical procedures. Traditional surgical techniques, while effective, often rely on manual manipulation and direct line-of-sight visualization, which can limit the potential accuracy and complexity of certain operations. In contrast, robotic surgical systems integrate advanced robotic technology with real-time imaging and computer-assisted guidance to facilitate surgery with improved outcomes.

Current robotic surgical systems utilize robotic arms controlled by surgeons from a console, offering articulated movements and ergonomic benefits that enable intricate maneuvers within the surgical field. By augmenting the abilities of the surgeon with higher accuracy, consistency, and intake of data, robotic surgical systems have the potential to both improve the safety and efficiency of surgical procedures, as well as reduce post-operation recovery times and risk of further complications.

Current robotic assisted surgical systems may have limited compatibility with instruments and implants of varied sizes, features, and configurations. As a result, the instruments and implants available to a surgeon during a specific procedure may not be optimal for a specific patient based on the patient's anatomy and clinical indications. To address these shortcomings, there is a need for a surgical system designed to accommodate a wider range of surgical instruments and implants.

### SUMMARY

The various systems and methods of the present disclosure have been developed in response to the present state of the art, and in particular, in response to the problems and needs in the art that have not yet been fully solved by currently available robotic assisted surgical systems and methods for spinal surgery.

In some embodiments, a surgical system configured for placement of a surgical screw into a bone may include the surgical screw having a screw head having a screw head diameter, a first arm, a second arm, and an inside thread, and a screw body coupled with the screw head and having a drive feature and a threaded portion configured to engage the bone. The system may further include a driver having a distal end configured to engage the surgical screw and a proximal end opposite the distal end, an outer shaft having a threaded portion configured to engage the inside thread, an inner shaft having a drive tip configured to engage the drive feature, and a sleeve configured to be received within an end effector of a robotic assisted surgical device, the sleeve having an outside diameter and an inside diameter. The outside diameter may be greater than or equal to the screw head diameter, the inside diameter may be less than the screw head diameter, and the sleeve may be further configured to receive the first arm and the second arm.

In the system of any preceding paragraph, the outside diameter may be configured to provide a bearing surface shaped to contact the end effector.

In the system of any preceding paragraph, the system may further include a thumb wheel configured to be removably receivable on the proximal end of the driver and engageable with the outer shaft so that rotation of the thumb wheel may result in rotation of the outer shaft, wherein the outer shaft may be configured to rotate independently of the sleeve to facilitate threaded engagement and threaded disengagement of the surgical screw.

In the system of any preceding paragraph, the system may further include an array configured to facilitate relative motion tracking of the surgical system, wherein the array may be configured to be received on the proximal end of the driver and secured to the outer shaft.

In the system of any preceding paragraph, the system may further include a handle configured to be removably receivable on the proximal end of the driver and engageable with the inner shaft so that rotation of the handle may result in rotation of the inner shaft.

In the system of any preceding paragraph, the sleeve may further include a third arm and a fourth arm, wherein the third arm and the fourth arm may be configured to be received between the first arm and the second arm.

In the system of any preceding paragraph, the inner shaft may be configured to rotate independently of the outer shaft and the sleeve.

In some embodiments, a surgical system configured for placement of a surgical screw into a bone may include a driver having a distal end configured to engage the surgical screw and a proximal end opposite the distal end, a sleeve configured to be received within an end effector of a robotic assisted surgical device, the sleeve having an outside diameter and an inside diameter, an outer shaft configured to be received in the sleeve and having a threaded portion configured to threadably engage the surgical screw, and an inner shaft configured to be received in the outer shaft and rotatable independently of the outer shaft. The system may further include an array configured to facilitate relative motion tracking of the surgical system. The outside diameter may be configured to provide a bearing surface shaped to contact the end effector, and the array may be configured to be received on the proximal end of the driver and secured to the outer shaft.

In the system of any preceding paragraph, the system may further include the surgical screw having a screw head including a screw head diameter, and a screw body coupled with the screw head and configured to engage the bone. The outside diameter may be greater than or equal to the screw head diameter, and the inside diameter may be less than the screw head diameter.

In the system of any preceding paragraph, the screw head may further include a first arm and a second arm and the sleeve may be further configured to receive the first arm and the second arm.

In the system of any preceding paragraph, the system may further include a thumb wheel configured to be removably receivable on the proximal end of the driver and engageable with the outer shaft so that rotation of the thumb wheel may result in rotation of the outer shaft, wherein the outer shaft may be configured to rotate independently of the sleeve to facilitate threaded engagement and threaded disengagement of the surgical screw.

In the system of any preceding paragraph, the system may further include a handle configured to be removably receivable on the proximal end of the driver and engageable with the inner shaft so that rotation of the handle may result in rotation of the inner shaft.

In the system of any preceding paragraph, the sleeve may further include a third arm and a fourth arm and the sleeve may further include a first arm and a second arm, wherein the third arm and the fourth arm may be configured to be received between the first arm and the second arm.

In the system of any preceding paragraph, the system may further include the surgical screw having a screw head having a second arm length and a screw body coupled to the screw head, wherein the sleeve may further include a first arm length, wherein the first arm length may be less than or equal to the second arm length.

In some embodiments, a surgical system configured for placement of a surgical screw into a bone may include the surgical screw having a screw head having a first arm, a second arm, and an inside thread, and a screw body coupled with the screw head and having a drive feature and a threaded portion configured to engage the bone. The system may further include a driver having a distal end configured to engage the surgical screw and a proximal end opposite the distal end, an outer shaft having a threaded portion configured to engage the inside thread, an inner shaft having a drive tip configured to engage the drive feature, and a sleeve configured to be received within an end effector of a robotic assisted surgical device, the sleeve having a third arm and a fourth arm. The third arm and the fourth arm may be configured to be received between the first arm and the second arm.

In the system of any preceding paragraph, the screw head may further include a screw head diameter. The sleeve may further include an outside diameter and an inside diameter, the outside diameter may be greater than or equal to the screw head diameter, the inside diameter may be less than the screw head diameter, and the sleeve may be further configured to receive the first arm and the second arm.

In the system of any preceding paragraph, the system may further include a thumb wheel configured to be removably receivable on the proximal end of the driver and engageable with the outer shaft so that rotation of the thumb wheel may result in rotation of the outer shaft, wherein the outer shaft may be configured to rotate independently of the sleeve to facilitate threaded engagement and threaded disengagement of the surgical screw.

In the system of any preceding paragraph, the system may further include a handle configured to be removably receivable on the proximal end of the driver and engageable with the inner shaft so that rotation of the handle may result in rotation of the inner shaft.

In the system of any preceding paragraph, the sleeve may further include an outside diameter, wherein the outside diameter may be configured to provide a bearing surface shaped to contact the end effector.

In the system of any preceding paragraph, the system may further include an array configured to facilitate relative motion tracking of the surgical system, wherein the array may be configured to be received on the proximal end of the driver and secured to the outer shaft.

These and other features and advantages of the present disclosure will become more fully apparent from the following description and appended claims or may be learned by the practice of the implants, systems, and methods set forth hereinafter.

### BRIEF DESCRIPTION OF THE DRAWINGS

Exemplary embodiments of the disclosure will become more fully apparent from the following description and appended claims, taken in conjunction with the accompanying drawings. Understanding that these drawings depict only exemplary embodiments and are, therefore, not to be considered limiting of the scope of the appended claims, the exemplary embodiments of the disclosure will be described with additional specificity and detail through use of the accompanying drawings in which:

FIG. 1A is a perspective view of a surgical system, including a driver, a handle, a surgical screw and an array, according to an embodiment;

FIG. 1B is a bottom perspective section view of the surgical system of FIG. 1A;

FIG. 2 is a perspective exploded view of the surgical system of FIG. 1A;

FIG. 3A is a perspective exploded view of the driver and array of FIG. 1A;

FIG. 3B is a perspective view of the driver and array of FIG. 1A;

FIG. 4A is a perspective view of the driver and array of FIG. 1A;

FIG. 4B is a front view of the driver and array of FIG. 1A;

FIG. 4C is a bottom view of the driver and array of FIG. 1A;

FIG. 5A is a perspective view of the handle of FIG. 1A;

FIG. 5B is a top view of the handle of FIG. 5A;

FIG. 5C is a front view of the handle of FIG. 5A;

FIG. 5D is a side view of the handle of FIG. 5A;

FIG. 6A is a perspective view of an inner shaft of the driver of FIG. 1A according to an embodiment;

FIG. 6B is a front view of the inner shaft of FIG. 6A;

FIG. 6C is a side view of the inner shaft of FIG. 6A;

FIG. 7A is a perspective view of an outer shaft of the driver of FIG. 1A according to an embodiment;

FIG. 7B is a section view of the outer shaft of FIG. 7A;

FIG. 7C is a front view of the outer shaft of FIG. 7A;

FIG. 7D is a side view of the outer shaft of FIG. 7A;

FIG. 8A is a perspective view of a tip of the driver of FIG. 1A according to an embodiment;

FIG. 8B is a section view of the tip of FIG. 8A;

FIG. 8C is a front view of the tip of FIG. 8A;

FIG. 8D is a side view of the tip of FIG. 8A;

FIG. 9A is a perspective view of a sleeve of the driver of FIG. 1A according to an embodiment;

FIG. 9B is a top view of the sleeve of FIG. 9A;

FIG. 9C is a front view of the sleeve of FIG. 9A;

FIG. 9D is a side view of the sleeve of FIG. 9A;

FIG. 10A is a perspective view of a thumb wheel of the driver of FIG. 1A according to an embodiment.

FIG. 10B is a section view of the thumb wheel of FIG. 10A;

FIG. 10C is a front view of the thumb wheel of FIG. 10A;

FIG. 10D is a side view of the thumb wheel of FIG. 10A;

FIG. 11A is a perspective view of a canted coil spring of the driver of FIG. 1A according to an embodiment;

FIG. 11B is a front view of the canted coil spring of FIG. 11A;

FIG. 11C is a side view of the canted coil spring of FIG. 11A;

FIG. 12A is a bottom perspective view of the array of FIG. 1A;

FIG. 12B is a perspective view of the array of FIG. 12A;

FIG. 12C is a top view of the array of FIG. 12A;

FIG. 12D is a section view of the array of FIG. 12A;

FIG. 12E is a front view of the array of FIG. 12A;

FIG. 12F is a side view of the array of FIG. 12A;

FIG. 13A is a perspective view of the surgical screw of FIG. 1A;

FIG. 13B is a top view of the surgical screw of FIG. 13A;

FIG. 13C is a front view of the surgical screw of FIG. 13A;

FIG. 13D is a side view of the surgical screw of FIG. 13A;

FIG. 14A is a perspective view of a driver, an array and a surgical screw according to an embodiment;

FIG. 14B is a perspective view of the driver without a thumb wheel, the array and the surgical screw of FIG. 14A;

FIG. 14C is a section view of the driver without a thumb wheel and the array of FIG. 14A;

FIG. 15A is a perspective view of a surgical system, including a driver, a handle, a surgical screw and an array, according to an embodiment;

FIG. 15B is a front section view of the driver of FIG. 15A;

FIG. 16A is a perspective view of a surgical system, including a driver, a handle, a surgical screw and an array, according to an embodiment;

FIG. 16B is a bottom perspective section view of the surgical system of FIG. 16A;

FIG. 17 is a perspective exploded view of the surgical system of FIG. 16A;

FIG. 18A is a perspective exploded view of the driver and array of FIG. 16A;

FIG. 18B is a perspective view of the driver and array of FIG. 16A;

FIG. 19A is a perspective view of the driver and array of FIG. 16A;

FIG. 19B is a front view of the driver and array of FIG. 16A;

FIG. 19C is a bottom view of the driver and array of FIG. 16A;

FIG. 20A is a perspective view of an inner shaft of the driver of FIG. 16A according to an embodiment;

FIG. 20B is a front view of the inner shaft of FIG. 20A;

FIG. 20C is a side view of the inner shaft of FIG. 20A;

FIG. 21A is a perspective view of an outer shaft of the driver of FIG. 16A according to an embodiment;

FIG. 21B is a section view of the outer shaft of FIG. 21A;

FIG. 21C is a front view of the outer shaft of FIG. 21A;

FIG. 21D is a side view of the outer shaft of FIG. 21A;

FIG. 22A is a perspective view of a tip of the driver of FIG. 16A according to an embodiment;

FIG. 22B is a section view of the tip of FIG. 22A;

FIG. 22C is a front view of the tip of FIG. 22A;

FIG. 22D is a side view of the tip of FIG. 22A;

FIG. 23A is a perspective view of a sleeve of the driver of FIG. 16A according to an embodiment;

FIG. 23B is a top view of the sleeve of FIG. 23A;

FIG. 23C is a front view of the sleeve of FIG. 23A;

FIG. 23D is a side view of the sleeve of FIG. 23A;

FIG. 24A is a perspective view of a collet of the driver of FIG. 16A according to an embodiment.

FIG. 24B is a section view of the collet of FIG. 24A;

FIG. 24C is a front view of the collet of FIG. 24A;

FIG. 24D is a side view of the collet of FIG. 24A;

FIG. 25A illustrates steps for assembling a surgical system according to an embodiment;

FIG. 25B is a perspective view of a surgical system, including a driver, a handle, a surgical screw and an array, according to an embodiment;

FIG. 25C is a perspective view of the surgical system of FIG. 25B;

FIG. 26 illustrates steps for assembling a surgical system according to an embodiment.

### DETAILED DESCRIPTION

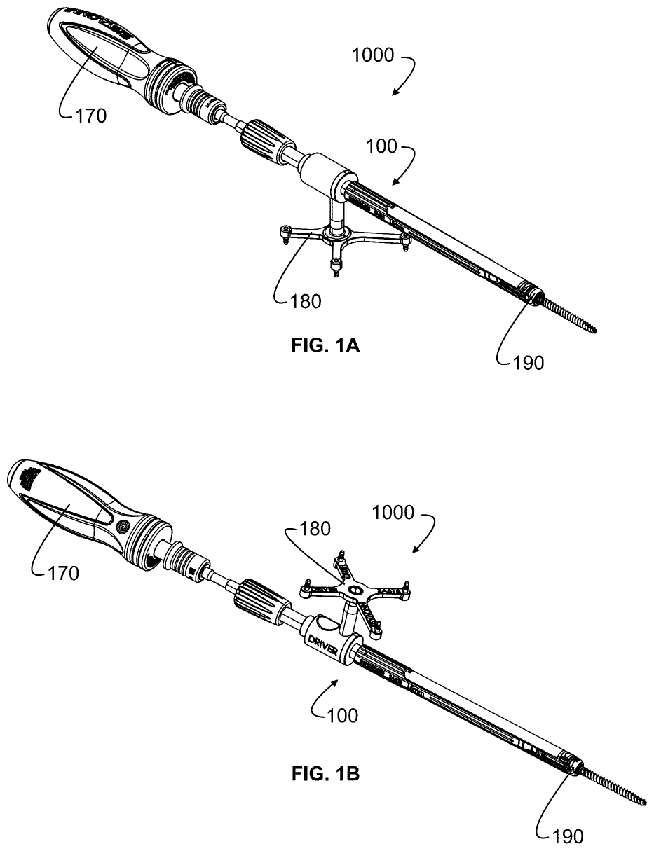

Exemplary embodiments of the disclosure will be best understood by reference to the drawings, wherein like parts are designated by like numerals throughout. It will be readily understood that the components of the disclosure, as generally described and illustrated in the Figures herein, could be arranged and designed in a wide variety of different configurations. Thus, the following more detailed description of the embodiments of the apparatus, system, and method, as represented in FIG. 1A through FIG. 26, is not intended to limit the scope of the claims, but is merely representative of exemplary embodiments of the present disclosure.

The phrases “connected to,” “coupled to” and “in communication with” refer to any form of interaction between two or more entities, including mechanical, electrical, magnetic, electromagnetic, fluid, and thermal interaction. Two components may be functionally coupled to each other even though they are not in direct contact with each other. The term “abutting” refers to items that are in direct physical contact with each other, although the items may not necessarily be attached together.

The word “exemplary” is used herein to mean “serving as an example, instance, or illustration.” Any embodiment described herein as “exemplary” is not necessarily to be construed as preferred or advantageous over other embodiments. While the various aspects of the embodiments are presented in drawings, the drawings are not necessarily drawn to scale unless specifically indicated.

Not every feature of each embodiment is labeled in every figure in which that embodiment appears, in order to keep the figures clear. Similar reference numbers (for example, those that are identical except for the first numeral) may be used to indicate similar features in different embodiments.

The present disclosure illustrates surgical systems for driving a surgical screw for the purposes of illustrating the concepts of the present design. However, it will be understood that other variations and uses are contemplated including, but not limited to, devices for drilling, tapping, navigation, probing, etc.

FIG. 1A is a perspective view of a surgical system 1000, including a driver 100, a handle 170, a surgical screw 190 and an array 180, according to an embodiment. FIG. 1B is a bottom perspective section view of the surgical system 1000. The surgical system 1000 may be configured to be coupled to a surgical instrument holding device. The surgical instrument holding device may be a robotic assisted surgical system, a surgical navigation system, and/or a minimally invasive surgical system. The surgical instrument holding device may include an engagement mechanism to receive the surgical system 1000. The engagement mechanism may include an aperture configured to receive the surgical system 1000. Additionally, or alternatively, the engagement mechanism may be configured as an end effector of a robotic arm as part of a robotic assisted surgical system. The surgical system 1000 may be configured to be received within the end effector of the robotic assisted surgical system. The surgical system 1000 may be configured so that the surgical screw 190 may pass through the engagement mechanism of the end effector. The driver 100 may be configured to be coupled to the engagement mechanism of the end effector.

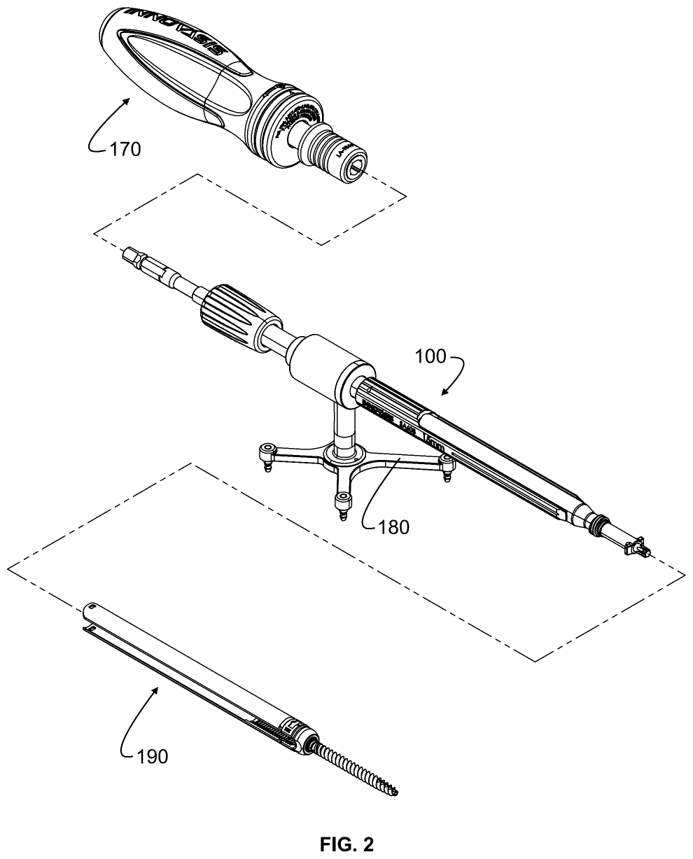

FIG. 2 is a perspective exploded view of the surgical system 1000. The handle 170 may be configured to be coupled to a proximal end of the driver 100. The driver 100 may further be configured to receive a surgical screw 190 at a distal end. The array 180 may be coupled to the driver 100. The driver 100 may be configured so that the driver 100, with the surgical screw 190 engaged, may be received within the array 180, the thumb wheel 150, and the handle 170.

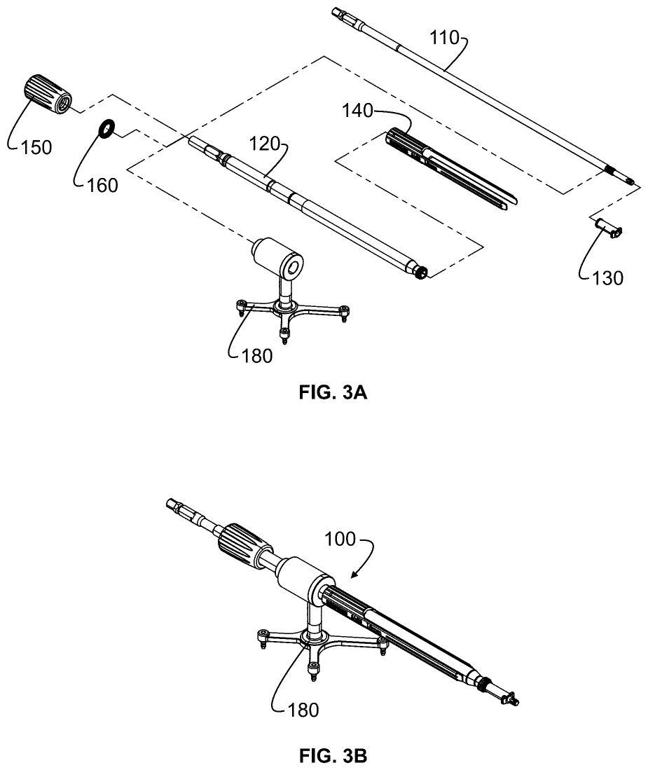

FIG. 3A is a perspective exploded view of the driver 100 and array 180. FIG. 3B is a perspective view of the driver 100 and array 180. The driver 100 may be configured to facilitate placement of a surgical screw 190 into a bone. More specifically, the driver 100 may be configured to facilitate placement of a pedicle screw into a pedicle of a vertebrae. More specifically, the driver 100 may be configured to facilitate placement of a poly-axial pedicle screw into a pedicle of a vertebrae. Additionally, or alternatively, the driver 100 may be configured to facilitate placement of a mono-axial pedicle screw into a pedicle of a vertebrae. Additionally, or alternatively, the driver 100 may be configured to facilitate placement of a uniplanar pedicle screw into a pedicle of a vertebrae.

The driver 100 may be configured to facilitate placement of a surgical screw 190 as part of a minimally invasive surgical procedure. Additionally, or alternatively, the driver 100 may be configured to facilitate placement of a surgical screw 190 as part of an open surgical procedure. Additionally, or alternatively, the driver 100 may be configured to facilitate placement of a surgical screw 190 as part of a robotically assisted surgical procedure. The driver 100 may include an inner shaft 110, an outer shaft 120, a tip 130, a sleeve 140, a thumb wheel 150, and a canted coil spring 160.

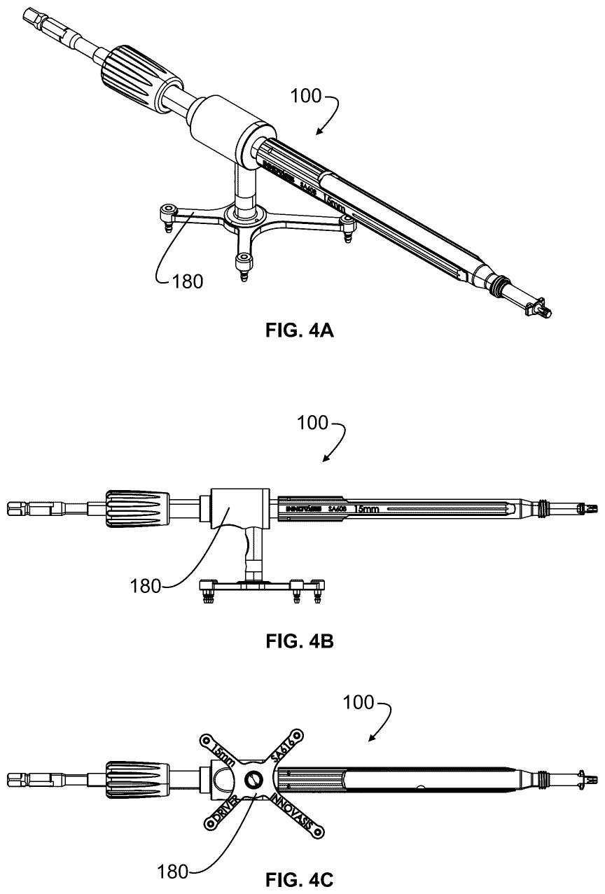

FIG. 4A is a perspective view of the driver 100 and the array 180. FIG. 4B is a front view of the driver 100 and the array 180. FIG. 4C is a bottom view of the driver 100 and the array 180. The driver 100 may be configured to removably engage a surgical screw 190 at a distal end. The driver 100 may further be configured to removably receive an array 180 and a thumb wheel 150 at a proximal end. Additionally, the proximal end of the driver 100 may be configured to be removably coupled with a handle 170.

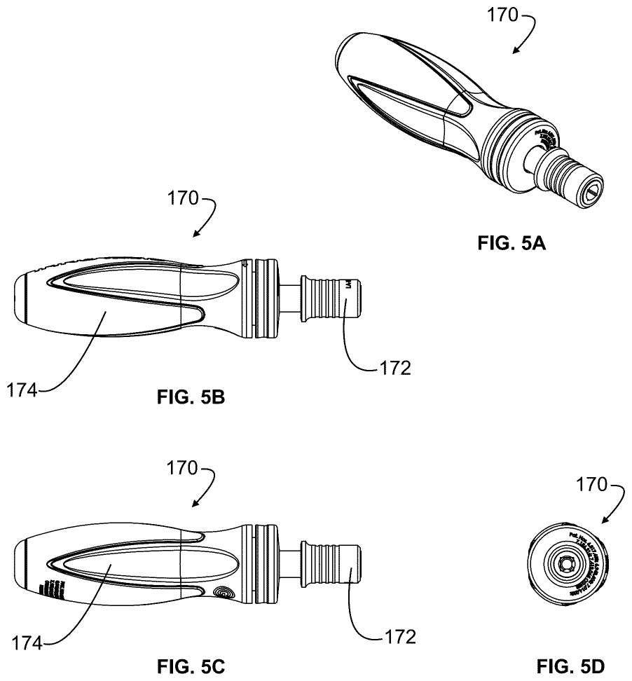

FIG. 5A is a perspective view of the handle 170, FIG. 5B is a top view of the handle 170, FIG. 5C is a front view of the handle 170, and FIG. 5D is a side view of the handle 170. The handle 170 may be configured to be removably coupled to the proximal end of the driver 100. The handle 170 may include a body 174 and a connector portion 172. The connector portion 172 may be configured to transfer rotation motion of the body 174 to rotational motion of the driver 100. The connector portion 172 may be configured with a female connection mechanism. Alternatively, the connector portion 172 may be configured with a male connection mechanism. The connector portion 172 may be configured with a quick connect style coupling mechanism. The connector portion 172 may be configured as one of: AO connector, Hudson connector, trilobe connector, square connector, hex connector, Stryker connector, Stryker-Hall connector, or other quick connect mechanism known in the art.

The body 174 of the handle 170 may be configured to allow a user to maintain a secure grip of the handle 170 and may facilitate rotation of the handle 170. The body 174 may include an ergonomic profile to facilitate a secure grip during rotation of the handle 170. Additionally, or alternatively, the body 174 may further include a textured surface to facilitate a secure grip during rotation of the handle 170. Additionally, or alternatively, the body 174 may be manufactured of medical grade silicone. Additionally, or alternatively, the body 174 may be made of a combination of materials including: silicone, nylon, ABS, polycarbonate, stainless steel, aluminum, other polymer, or other medical grade materials.

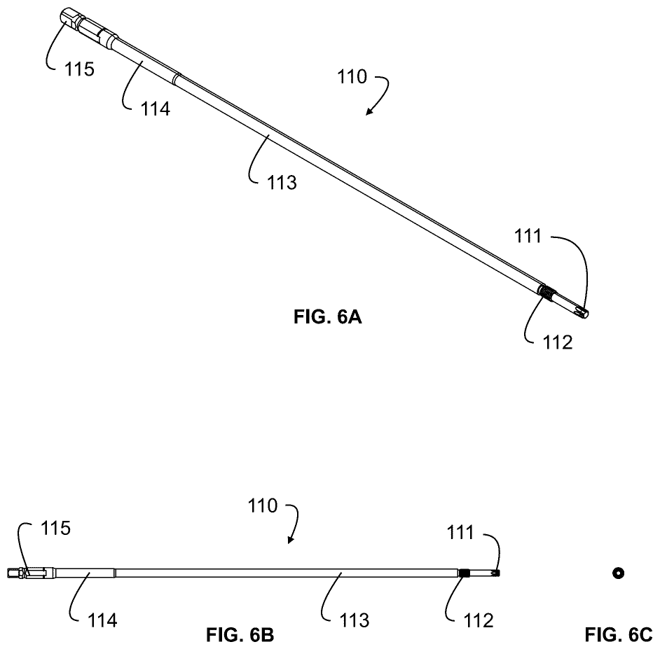

FIG. 6A is a perspective view of an inner shaft 110 of the driver 100 according to an embodiment. FIG. 6B is a front view of the inner shaft 110. FIG. 6C is a side view of the inner shaft 110. The inner shaft 110 may be configured to transfer rotational motion of the handle 170 to rotational motion of the surgical screw 190. The inner shaft 110 may include a drive tip 111, a threaded portion 112, a first shaft diameter 113, a second shaft diameter 114, and a connector portion 115.

The drive tip 111 may be configured to be received within a drive feature of the surgical screw 190. The drive tip 111 may be configured as one of: a hexalobe, a hex, a square, a trilobe, or other non-circular geometry. The drive tip 111 may be configured to transfer rotational motion of the inner shaft 110 to the surgical screw 190.

The second shaft diameter 114 may be configured to be slidably received within the first inside diameter 121 of the outer shaft 120. The first shaft diameter 113 may be configured to be less than or equal to the second shaft diameter 114. The second shaft diameter 114 may be greater than or equal to the first shaft diameter 113, the threaded portion 112 and the drive tip 111. Additionally, the second shaft diameter 114 may be less than or equal to the connector portion 115. Additionally, the connector portion 115 may be greater than the first inside diameter 121 of the outer shaft 120.

The threaded portion 112 may be configured to threadably engage the tip 130. The inner shaft 110 may be configured so that the inner shaft 110 may be slidably received within the outer shaft and the tip 130 may be threadably engaged with the inner shaft 110, whereby the outer shaft 120 is captive on the inner shaft 110. The connector portion 115 may be configured to be removably coupled with the connector portion 172 of the handle 170. The connector portion 115 may be configured to transfer rotational motion of the handle 170 to rotational motion of the inner shaft 110. The connector portion 115 may be configured as one of: AO connector, Hudson connector, trilobe connector, square connector, hex connector, Stryker connector, Stryker-Hall connector, or other quick connect mechanism known in the art.

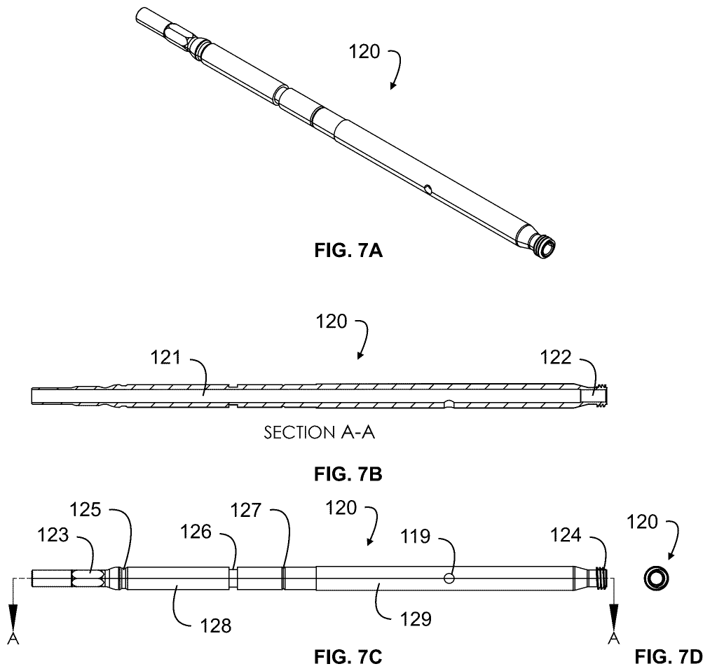

FIG. 7A is a perspective view of an outer shaft 120 of the driver 100 according to an embodiment. FIG. 7B is a section view of the outer shaft 120, FIG. 7C is a front view of the outer shaft 120, and FIG. 7D is a side view of the outer shaft 120. The outer shaft 120 may be configured to retain the surgical screw 190 on the inner shaft 110. Additionally, the outer shaft 120 may be configured to be received within the array 180 and the thumb wheel 150. The outer shaft 120 may include a first inside diameter 121, a second inside diameter 122, a drive portion 123, a threaded portion 124, a first retaining groove 125, a second retaining groove 126, a third retaining groove 127, a first outside diameter 128, a second outside diameter 129, and an aperture 119.

The first inside diameter 121 may be configured to slidably receive the inner shaft 110. The second inside diameter 122 may be less than or equal to the first inside diameter 121. The second inside diameter 122 may be configured to slidably receive the first shaft diameter 113. The first inside diameter 121 and the second shaft diameter 114 may be configured so that the inner shaft 110 and outer shaft 120 are coaxial. Additionally, the second inside diameter 122 and the first shaft diameter 113 may be configured so that the inner shaft 110 and outer shaft 120 are coaxial. The gap between the first shaft diameter 113 and the first inside diameter 121 may be configured to facilitate cleaning and/or flushing through the aperture 119.

The drive portion 123 may be configured to be received within the thumb wheel 150. The drive portion 123 may be configured to transfer rotational motion of the thumb wheel 150 to rotational motion of the outer shaft 120 in order to threadably engage the threaded portion 124 of the outer shaft 120 with the inside thread 194 of the surgical screw 190. The drive portion 123 may be configured as a hexagon, square, octagon, or other non-circular geometry. The drive portion 123 may be configured to couple with the thumb wheel 150 when the proximal end of the outer shaft 120 is received within the thumb wheel 150.

The first retaining groove 125 may be configured to receive the canted coil spring 160 to retain the thumb wheel 150 on the outer shaft 120. The second retaining groove 126 may be configured to receive a locking mechanism 182 of the array 180 to retain the array 180 on the outer shaft 120. The third retaining groove 127 may be configured to receive a dowel pin or other retention feature that engages a location feature 145 of the sleeve 140.

The first outside diameter 128 may be smaller than the inside diameter 181 of the array 180. The first outside diameter 128 may also be smaller than the first inside diameter 154 of the thumb wheel 150. Additionally, the first outside diameter 128 may be smaller than the second outside diameter 129. The first outside diameter 128 may be configured to be received within the thumb wheel 150 and the array 180. The second outside diameter 129 may be smaller than the inside diameter 141 of the sleeve 140. The second outside diameter 129 may be configured to be received within the sleeve 140.

The driver 100 may include a distal end configured to engage the surgical screw 190 and a proximal end opposite the distal end. The proximal end of the driver 100, including the second shaft diameter 114 and the connector portion 115 of the inner shaft 110 and the first outside diameter 128 and the drive portion 123 of the outer shaft 120, may be smaller than the inside diameter 181. In an embodiment, the inside diameter 181 may be approximately Ø11 mm and the second shaft diameter 114, the connector portion 115, the first outside diameter 128, and the drive portion 123 may each be less than or equal to Ø11 mm.

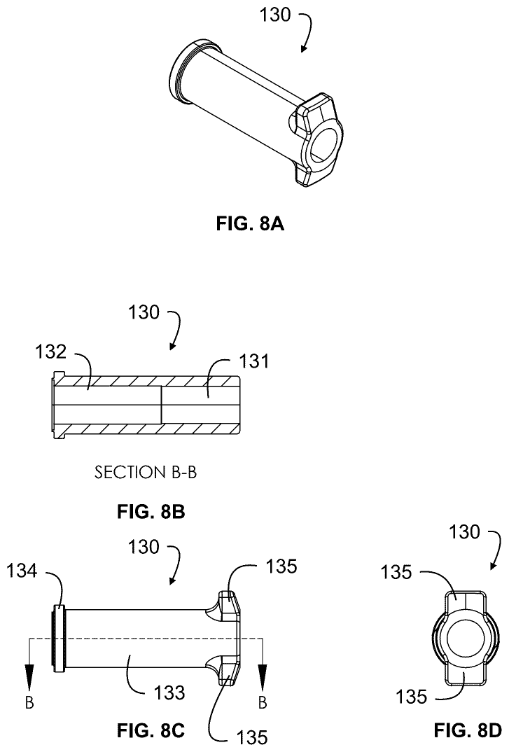

FIG. 8A is a perspective view of a tip 130 of the driver 100 according to an embodiment. FIG. 8B is a section view of the tip 130, FIG. 8C is a front view of the tip 130, and FIG. 8D is a side view of the tip 130. The tip 130 may be configured to engage the inner shaft 110 so that the outer shaft 120 is captive on the inner shaft 110. The tip 130 may be configured to engage the surgical screw 190 between a first arm 192 and a second arm 193. The tip 130 may further be configured to provide a counter-torque to the surgical screw 190 as the threaded portion 124 of the outer shaft 120 engages the inside thread 194 of the surgical screw 190.

The tip 130 may include a first inside diameter 131, a second inside diameter 132, an outside diameter 133, a flange 134, and one or more wings 135. The first inside diameter 131 may be larger than the drive tip 111 of the inner shaft 110. The first inside diameter 131 may be configured to slidably receive the drive tip 111. The second inside diameter 132 may be configured to securely engage the threaded portion 112 of the inner shaft 110. The second inside diameter 132 may be configured as an internal thread configured to threadably engage the threaded portion 112. The outside diameter 133 may be configured to be received between the first arm 192 and second arm 193 of the surgical screw 190 and may be smaller than a minor diameter of the threaded portion 124 of the outer shaft 120.

The flange 134 may be equal to or smaller than a minor diameter of the threaded portion 124 and larger than the second inside diameter 122 of the outer shaft 120 thereby preventing the outer shaft 120 from being removed from the inner shaft 110. Additionally, the flange 134 may not interfere with the threaded portion 124 of the outer shaft 120 from engaging the inside thread 194 of the surgical screw 190. The one or more wings 135 may be smaller than the distance between the first arm 192 and the second arm 193 of the surgical screw 190.

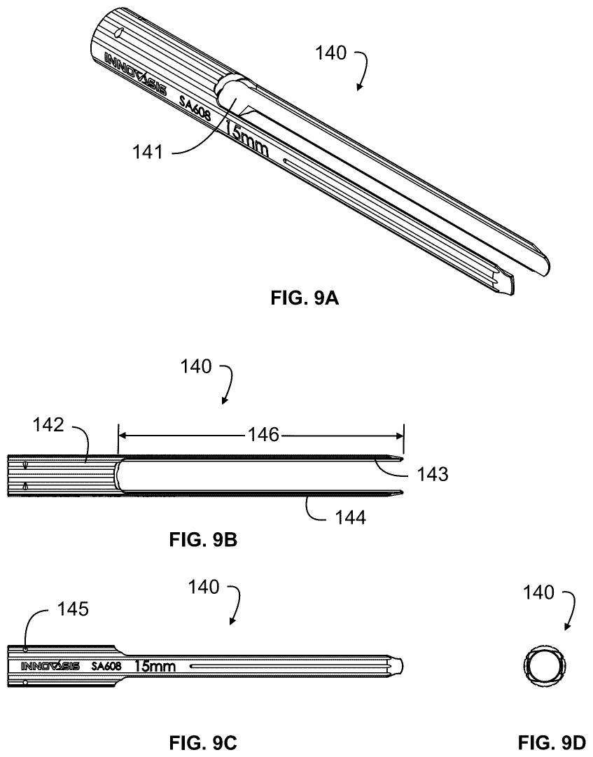

FIG. 9A is a perspective view of a sleeve 140 of the driver 100 according to an embodiment. FIG. 9B is a top view of the sleeve 140, FIG. 9C is a front view of the sleeve 140, and FIG. 9D is a side view of the sleeve 140. The sleeve 140 may be configured to be received within a surgical instrument holding device. The sleeve 140 may be configured to be received within an end effector of a robotic assisted surgical device.

The sleeve 140 may include an outside diameter 142. The outside diameter 142 may be greater than or equal to a screw head diameter 196 of the surgical screw 190. Additionally, the sleeve may include an inside diameter 141 that may be less than the screw head diameter 196.

The sleeve 140 may be sized to be slidably received within an aperture of an engagement mechanism of a surgical instrument holding device. The sleeve 140 may be configured to facilitate engagement of a surgical system 1000 with a surgical instrument holding device, thereby facilitating placement of a surgical screw 190, in which the screw head diameter 196 is smaller than the aperture of an engagement mechanism of the surgical instrument holding device, using the surgical instrument holding device.

The outside diameter 142 may be configured to provide a bearing surface shaped to contact an end effector. The sleeve 140 may be configured to rotate independently of the outer shaft 120 to facilitate threaded engagement and threaded disengagement of the surgical screw 190. More specifically, the outside diameter 142 may be configured to provide a bearing surface for a Ø15 mm end effector.

The sleeve 140 may further include an inside diameter 141, a third arm 143, a fourth arm 144, a location feature 145 and an arm length 146. The inside diameter 141 may be configured to slidably receive the second outside diameter 129 of the outer shaft 120. The third arm 143 may be configured to be received between the first arm 192 and the second arm 193 of the surgical screw 190. The fourth arm 144 may be configured to be received between the first arm 192 and the second arm 193 of the surgical screw 190. The arm length 146 may be less than the arm length 197 of the surgical screw 190. Alternatively, the arm length 146 may be greater than or equal to the arm length 197 of the surgical screw 190.

The location feature 145 may be configured to allow rotation of the sleeve 140 about the outer shaft 120 while preventing translation of the sleeve 140 along an axis. The location feature 145 may include one or more dowel pins configured to be received within a third retaining groove 127 of the outer shaft 120. Alternatively, the location feature 145 may include an o-ring, a snap ring, a retaining ring, a set screw, or other type of retention mechanism know in the art.

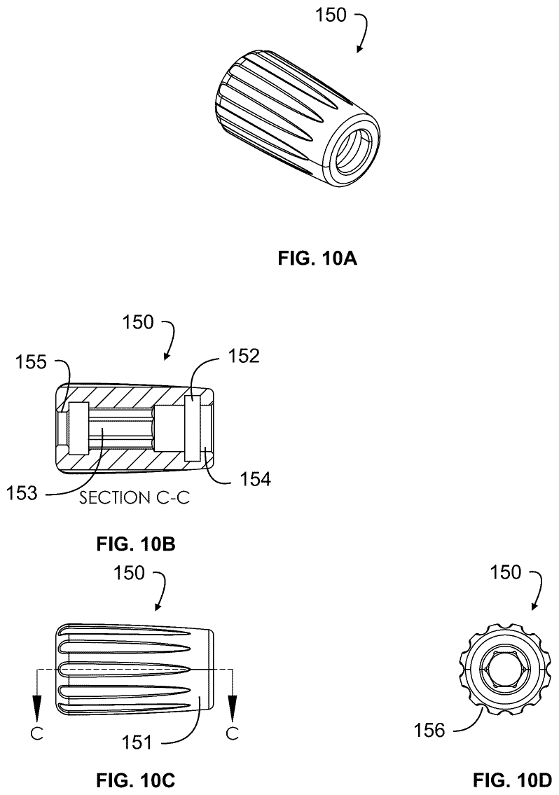

FIG. 10A is a perspective view of a thumb wheel 150 of the driver 100 according to an embodiment. FIG. 10B is a section view of the thumb wheel 150, FIG. 10C is a front view of the thumb wheel 150, and FIG. 10D is a side view of the thumb wheel 150. The thumb wheel 150 may be configured to slidably receive the outer shaft 120. The thumb wheel 150 may be configured so that rotation of the thumb wheel results in rotation of the outer shaft.

The thumb wheel 150 may include a drive portion 153 configured to engage the drive portion 123 of the outer shaft 120. The drive portion 153 may be configured to transfer rotational motion of the thumb wheel 150 to rotational motion of the outer shaft 120 to facilitate engagement of the threaded portion 124 of the outer shaft 120 with the inside thread 194 of the surgical screw 190. The drive portion 153 may be configured as a hexagon, square, octagon, or other non-circular geometry that corresponds to the geometry of the drive portion 123 of the outer shaft 120. In an embodiment, the drive portion 153 may be configured as an 8 mm female hex. The drive portion 153 may be configured to couple the thumb wheel 150 with the outer shaft 120 when the proximal end of the outer shaft 120 is received within the thumb wheel 150.

The thumb wheel 150 may also include an outside diameter 151, a retaining groove 152, a first inside diameter 154, a second inside diameter 155, and a grip feature 156. The outside diameter 151 may be sized and shaped ergonomically. More specifically, the outside diameter 151 may have a profile that ranges from 16 mm to 25 mm. Additionally, the outside diameter 151 may include a grip feature 156 configured to improve the grip of a user during rotational movement of the thumb wheel 150. The grip feature 156 may include a radial pattern of axial grooves. Alternatively, the grip feature 156 may include a knurled pattern on the outside diameter 151. Alternatively, the grip feature 156 may include a roughened surface treatment. Alternatively, the grip feature 156 may include a radial pattern of flat surfaces. Alternatively, the thumb wheel 150 may have a non-circular cross-sectional profile.

The retaining groove 152 may be configured to receive the canted coil spring 160 to retain the thumb wheel 150 on the outer shaft 120. The second inside diameter 155 may be configured to receive a proximal end of the outer shaft 120.

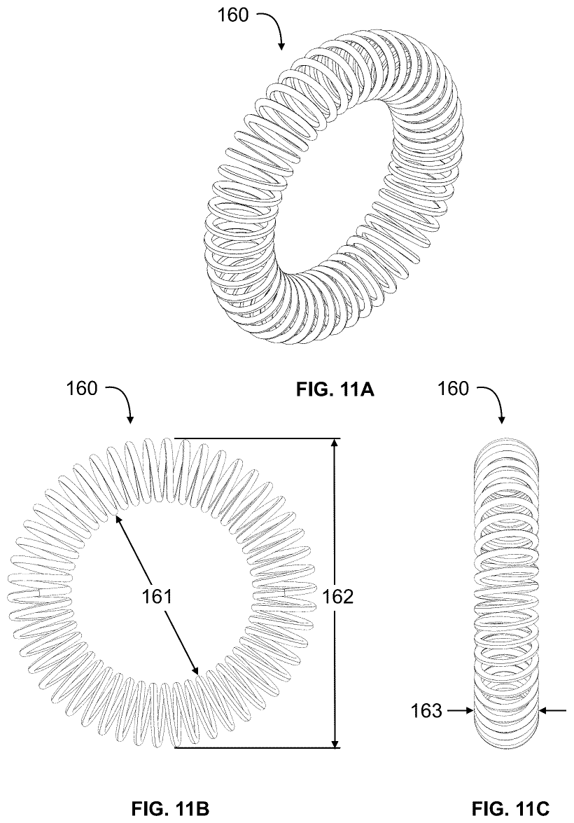

FIG. 11A is a perspective view of a canted coil spring 160 of the driver 100 according to an embodiment. FIG. 11B is a front view of the canted coil spring 160 and FIG. 11C is a side view of the canted coil spring 160. The canted coil spring 160 may be configured to allow the thumb wheel 150 to removably engage the outer shaft 120. The canted coil spring 160 may include an inside diameter 161, an outside diameter 162 and a width 163. The outside diameter 162 may be configured to be seated within the retaining groove 152 of the thumb wheel 150. The inside diameter 161 may be configured to be seated within the first retaining groove 125 of the outer shaft 120. The width 163 may be configured so that the canted coil spring 160 may be received within the retaining groove 152 and the first retaining groove 125.

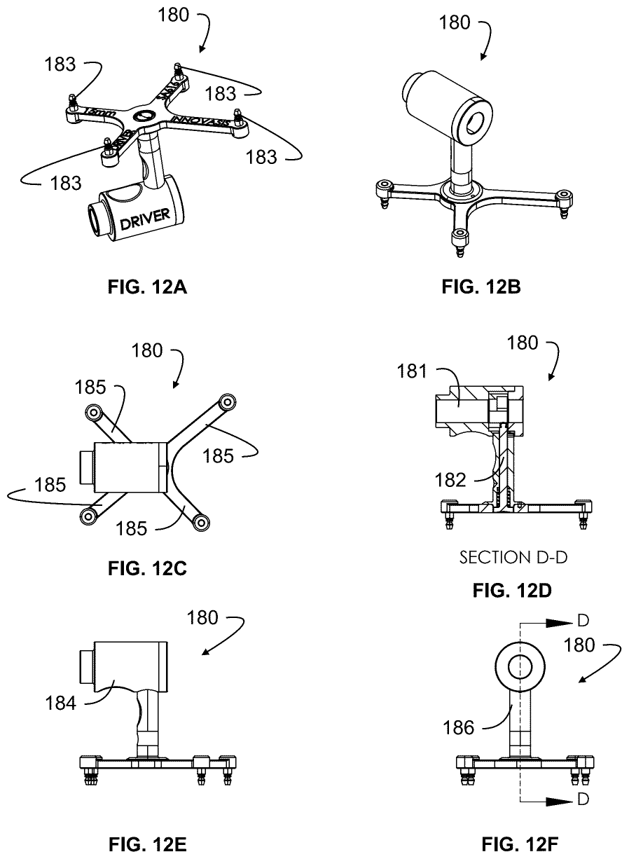

FIG. 12A is a bottom perspective view of the array 180, FIG. 12B is a perspective view of the array 180, FIG. 12C is a top view of the array 180, FIG. 12D is a section view of the array 180, FIG. 12E is a front view of the array 180, and FIG. 12F is a side view of the array 180. The array 180 may be configured to receive the driver 100, specifically the outer shaft 120 of the driver 100. The array 180 may be configured to facilitate real-time trajectory alignment and relative motion tracking of the surgical system 1000 by a surgical navigation system and/or a robotic assisted surgical system.

The array 180 may include an inside diameter 181, a locking mechanism 182, a plurality of locators 183, a body 184, a plurality of arms 185, and a post 186. The inside diameter 181 may be configured to receive the first outside diameter 128 of the outer shaft 120. The locking mechanism 182 may be configured to secure the array 180 to the driver 100, more specifically, to the outer shaft 120. The locking mechanism 182 may include a threaded component configured to engage the second retaining groove 126 of the outer shaft 120 so that the array 180 is prevented from moving in relation to the outer shaft 120.

The plurality of locators 183 may be positioned at the ends of the plurality of arms 185. The plurality of locators 183 may be configured as reference features to facilitate real-time trajectory alignment and relative motion tracking of the surgical system 1000 by a surgical navigation system and/or a robotic assisted surgical system. The body 184 may include the inside diameter 181. The post 186 may connect the body 184 to the plurality of arms 185. The locking mechanism 182 may extend through the post 186 into the body 184 and into the inside diameter 181.

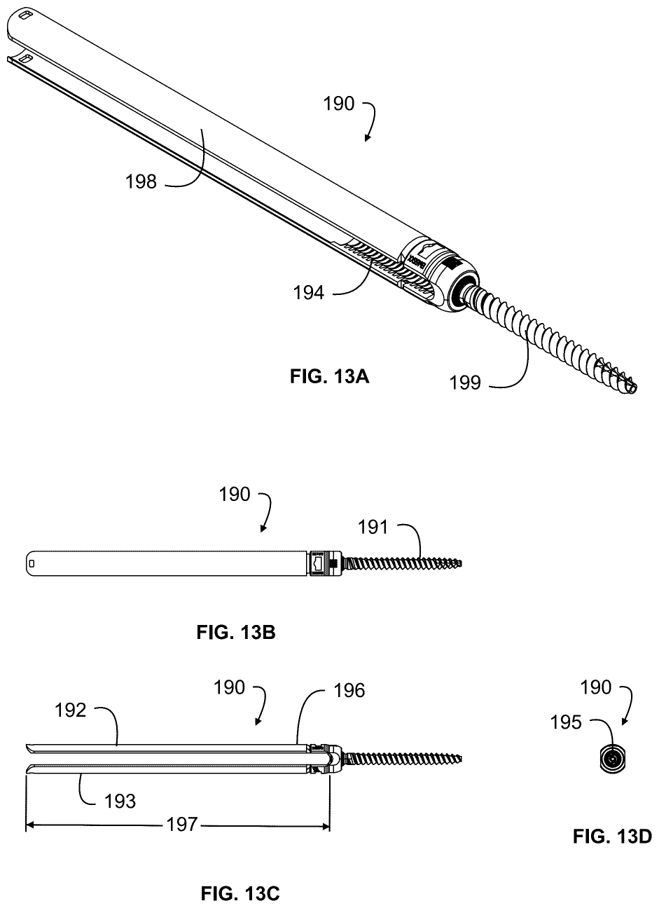

FIG. 13A is a perspective view of the surgical screw 190, FIG. 13B is a top view of the surgical screw 190, FIG. 13C is a front view of the surgical screw 190, and FIG. 13D is a side view of the surgical screw 190. The surgical screw 190 may be configured for placement into a bone. The surgical screw 190 may further be configured to be placed using a surgical navigation system and/or a robotic assisted surgical system. Additionally, or alternatively, the surgical screw 190 may be configured to be placed into a bone using a minimally invasive surgical technique.

The surgical screw 190 may include a screw head 198 and a screw body 199 coupled with the screw head 198. The screw head 198 may include a first arm 192, a second arm 193, an inside thread 194, an arm length 197, and screw head diameter 196. The screw body 199 may include a threaded portion 191, and a drive feature 195. The threaded portion 191 may be configured to engage a bone and may include a cancellous thread form and/or a cortical thread form.

The drive feature 195 may be configured to receive the drive tip 111 of the inner shaft 110. The drive feature 195 may be configured as one of: a hexalobe, a hex, a square, a trilobe, or other non-circular geometry. The drive feature 195 may be configured with a geometry corresponding to the geometry of the drive tip 111.

The inside thread 194 may be configured to threadably receive the threaded portion 124 of the outer shaft 120. The inside thread 194 may further be configured to receive a set screw (not shown) after placement of the threaded portion 191 into a bone. The inside thread 194 may be within an inside diameter defined by the first arm 192 and the second arm 193.

The first arm 192 and the second arm 193 may be configured to receive the third arm 143 and the fourth arm 144 of the sleeve 140 therebetween. The first arm 192 and the second arm 193 may define a screw head diameter 196. The screw head diameter 196 may be equal to or smaller than the outside diameter 142 of the sleeve 140. The screw head diameter 196 may be greater than the inside diameter 141 of the sleeve 140. The arm length 197 may be greater than or equal to the arm length 146 of the sleeve 140. Alternatively, the arm length 197 may be less than the arm length 146 of the sleeve 140.

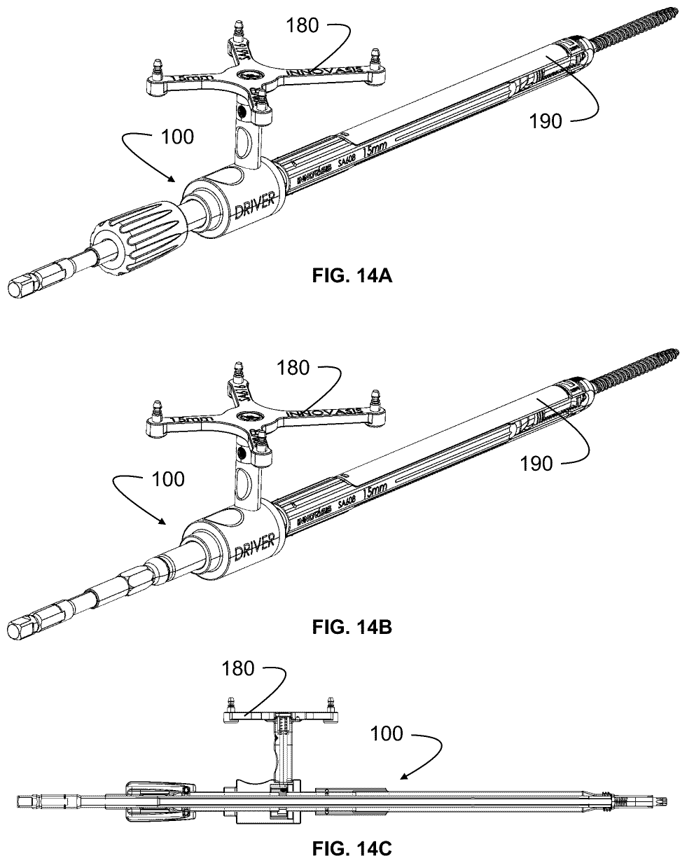

FIG. 14A is a perspective view of a driver 100, an array 180 and a surgical screw 190 according to an embodiment. FIG. 14B is a perspective view of the driver 100 without a thumb wheel 150, the array 180 and the surgical screw 190. FIG. 14C is a section view of the driver 100 without a thumb wheel 150 and the array 180. The array 180 may be configured to removably receive the outer shaft 120 of the driver 100. Additionally, the thumb wheel 150 may be configured to removably receive the outer shaft 120 of the driver 100.

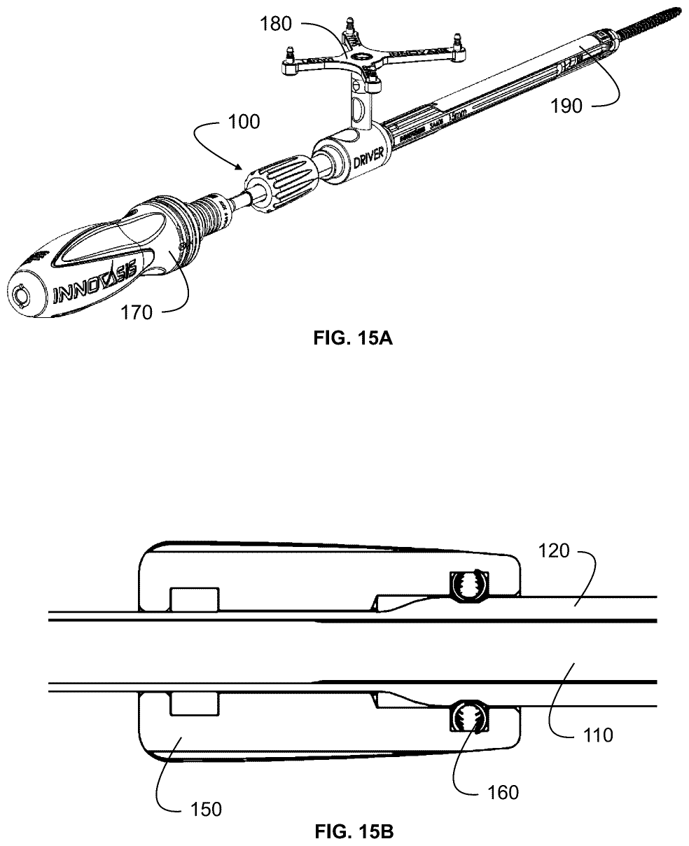

FIG. 15A is a perspective view of a surgical system 1000, including a driver 100, a handle 170, a surgical screw 190 and an array 180, according to an embodiment. FIG. 15B is a front section view of the driver 100. The handle 170 may be configured to removably couple with the inner shaft 110 of the driver 100. The handle 170 may be configured so that rotation of the handle results in rotation of the inner shaft. The canted coil spring 160 may be configured to facilitate removeable engagement of the thumb wheel 150 with the outer shaft 120. The canted coil spring 160 may be received within the first retaining groove 125 of the outer shaft 120 and the retaining groove 152 of the thumb wheel 150.

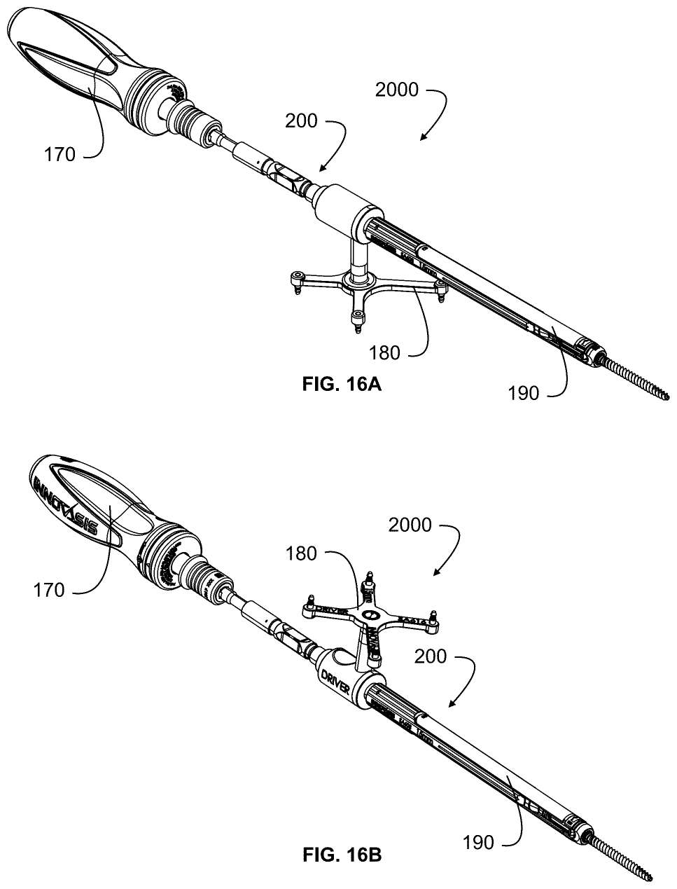

FIG. 16A is a perspective view of a surgical system 2000, including a driver 200, a handle 170, a surgical screw 190 and an array 180, according to an embodiment. FIG. 16B is a bottom perspective section view of the surgical system 2000. The surgical system 2000 may be configured to be coupled to a surgical instrument holding device. The surgical instrument holding device may be a robotic assisted surgical system, a surgical navigation system, and/or a minimally invasive surgical system. The surgical instrument holding device may include an engagement mechanism to receive the surgical system 2000. The engagement mechanism may include an aperture configured to receive the surgical system 2000. Additionally, or alternatively, the engagement mechanism may be configured as an end effector of a robotic arm as part of a robotic assisted surgical system. The surgical system 2000 may be configured to be received within the end effector of the robotic assisted surgical system. The surgical system 2000 may be configured so that the surgical screw 190 may pass through the engagement mechanism of the end effector. The driver 200 may be configured to be coupled to the engagement mechanism of the end effector.

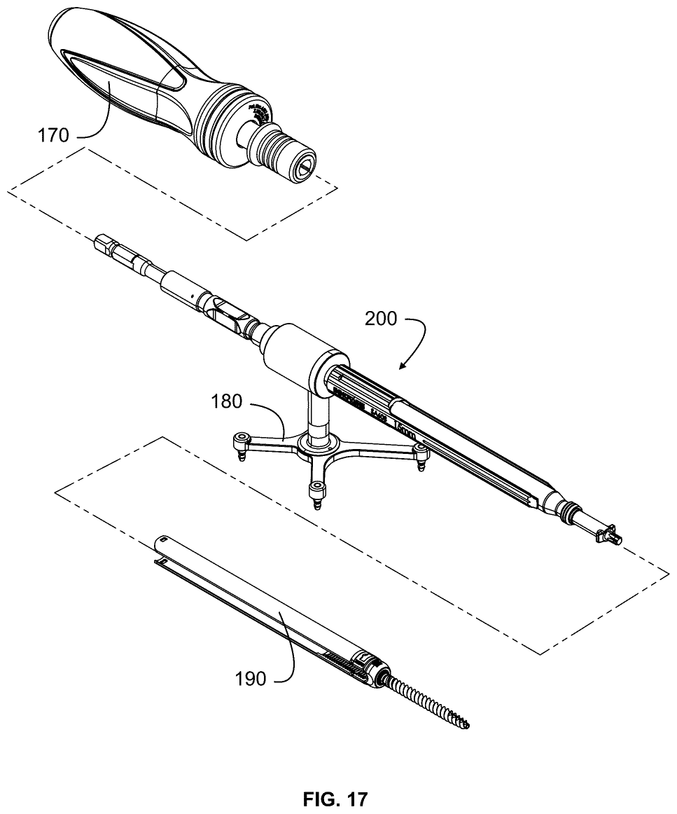

FIG. 17 is a perspective exploded view of the surgical system 2000. The handle 170 may be configured to be coupled to a proximal end of the driver 200. The driver 200 may further be configured to receive a surgical screw 190 at a distal end. The array 180 may be coupled to the driver 200. The driver 200 may be configured so that the driver 200, with the surgical screw 190 engaged, may be received within the array 180, the collet 250, and the handle 170.

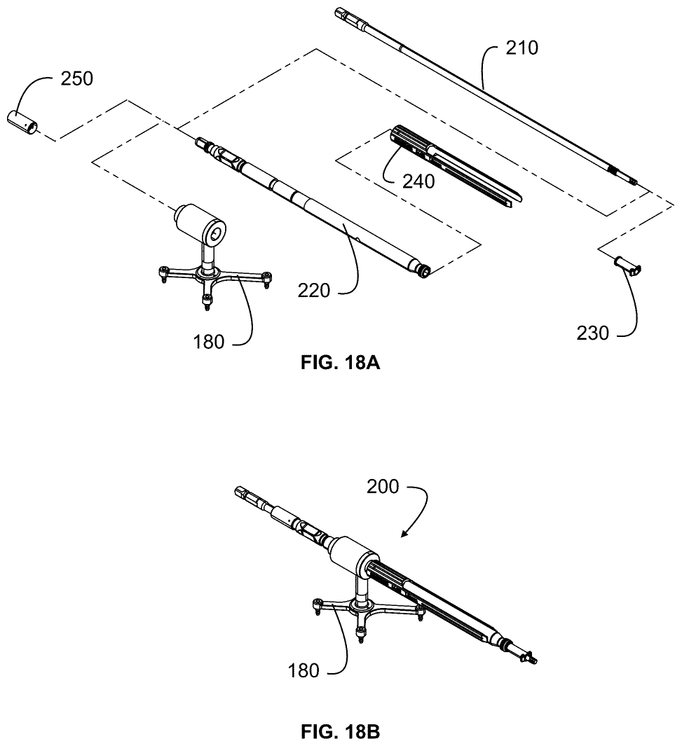

FIG. 18A is a perspective exploded view of the driver 200 and array 180. FIG. 18B is a perspective view of the driver 200 and array 180. The driver 200 may be configured to facilitate placement of a surgical screw 190 into a bone. More specifically, the driver 200 may be configured to facilitate placement of a pedicle screw into a pedicle of a vertebrae. More specifically, the driver 200 may be configured to facilitate placement of a poly-axial pedicle screw into a pedicle of a vertebrae. Additionally, or alternatively, the driver 200 may be configured to facilitate placement of a mono-axial pedicle screw into a pedicle of a vertebrae. Additionally, or alternatively, the driver 200 may be configured to facilitate placement of a uniplanar pedicle screw into a pedicle of a vertebrae.

The driver 200 may be configured to facilitate placement of a surgical screw 190 as part of a minimally invasive surgical procedure. Additionally, or alternatively, the driver 200 may be configured to facilitate placement of a surgical screw 190 as part of an open surgical procedure. Additionally, or alternatively, the driver 200 may be configured to facilitate placement of a surgical screw 190 as part of a robotically assisted surgical procedure. The driver 200 may include an inner shaft 210, an outer shaft 220, a tip 230, a sleeve 240, and a collet 250.

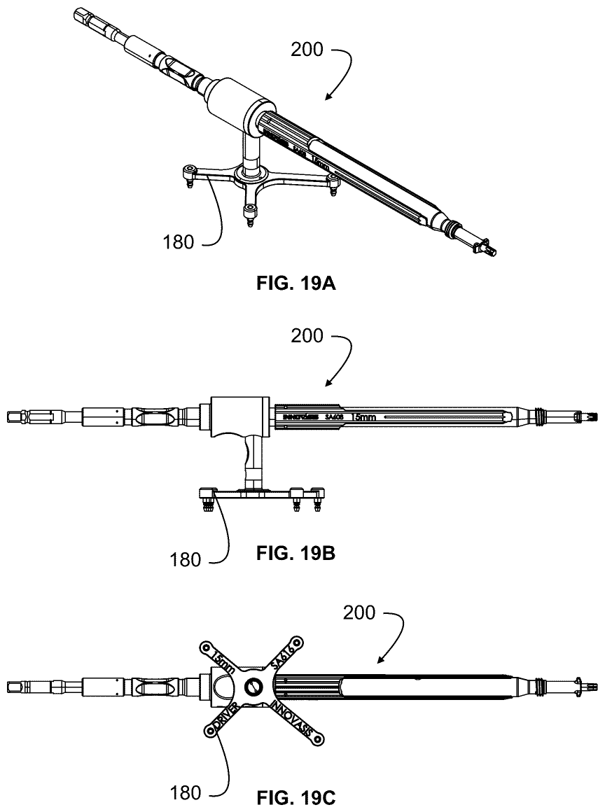

FIG. 19A is a perspective view of the driver 200 and the array 180. FIG. 19B is a front view of the driver 200 and the array 180. FIG. 19C is a bottom view of the driver 200 and the array 180. The driver 200 may be configured to removably engage a surgical screw 190 at a distal end. The driver 200 may further be configured to removably receive an array 180 and a collet 250 at a proximal end. Additionally, the proximal end of the driver 200 may be configured to be removably coupled with a handle 170.

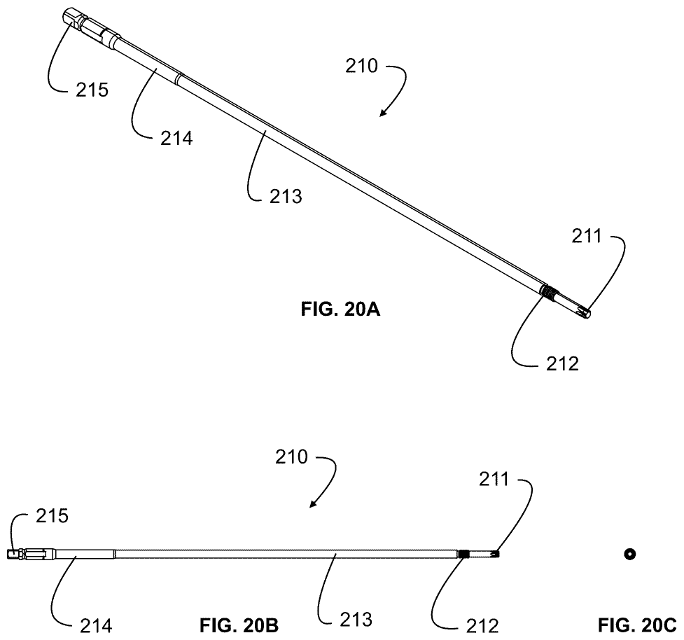

FIG. 20A is a perspective view of an inner shaft 210 of the driver 200 according to an embodiment. FIG. 20B is a front view of the inner shaft 210. FIG. 20C is a side view of the inner shaft 210. The inner shaft 210 may be configured to transfer rotational motion of the handle 170 to rotational motion of the surgical screw 190. The inner shaft 210 may include a drive tip 211, a threaded portion 212, a first shaft diameter 213, a second shaft diameter 214, and a connector portion 215.

The drive tip 211 may be configured to be received within a drive feature of the surgical screw 190. The drive tip 211 may be configured as one of: a hexalobe, a hex, a square, a trilobe, or other non-circular geometry. The drive tip 211 may be configured to transfer rotational motion of the inner shaft 210 to the surgical screw 190.

The second shaft diameter 214 may be configured to be slidably received within the first inside diameter 221 of the outer shaft 220. The first shaft diameter 213 may be configured to be less than or equal to the second shaft diameter 214. The second shaft diameter 214 may be greater than or equal to the first shaft diameter 213, the threaded portion 212 and the drive tip 211. Additionally, the second shaft diameter 214 may be less than or equal to the connector portion 215. Additionally, the connector portion 215 may be greater than the first inside diameter 221 of the outer shaft 220.

The threaded portion 212 may be configured to threadably engage the tip 230. The inner shaft 210 may be configured so that the inner shaft 210 may be slidably received within the outer shaft and the tip 230 may be threadably engaged with the inner shaft 210, whereby the outer shaft 220 is captive on the inner shaft 210. The connector portion 215 may be configured to be removably coupled with the connector portion 172 of the handle 170. The connector portion 215 may be configured to transfer rotational motion of the handle 170 to rotational motion of the inner shaft 210. The connector portion 215 may be configured as one of: AO connector, Hudson connector, trilobe connector, square connector, hex connector, Stryker connector, Stryker-Hall connector, or other quick connect mechanism known in the art.

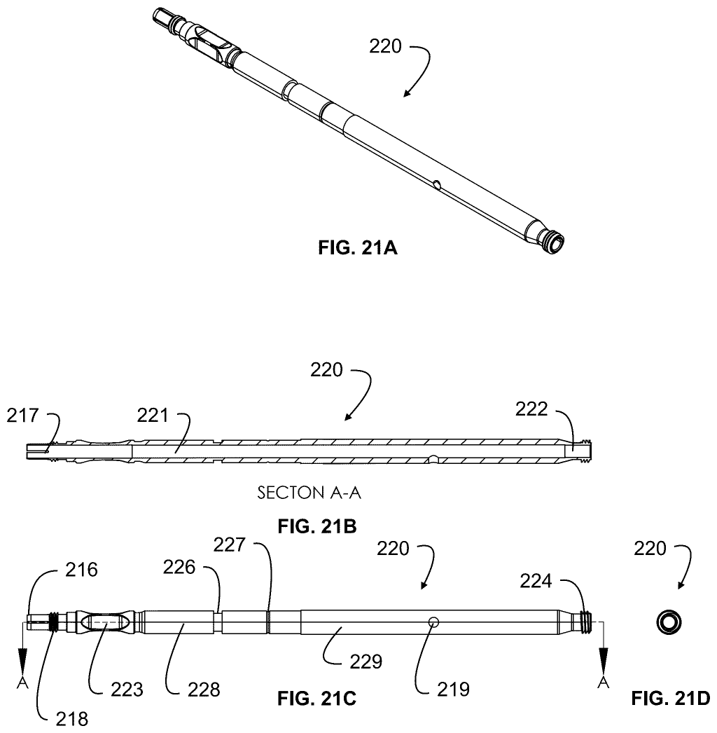

FIG. 21A is a perspective view of an outer shaft 220 of the driver 200 according to an embodiment. FIG. 21B is a section view of the outer shaft 220, FIG. 21C is a front view of the outer shaft 220, and FIG. 21D is a side view of the outer shaft 220. The outer shaft 220 may be configured to retain the surgical screw 190 on the inner shaft 210. Additionally, the outer shaft 220 may be configured to be received within the array 180 and the collet 250. The outer shaft 220 may include a first inside diameter 221, a second inside diameter 222, a grip portion 223, a first threaded portion 224, a second retaining groove 226, a third retaining groove 227, a first outside diameter 228, a second outside diameter 229, an aperture 219, a second threaded portion 218, one or more slots 217, and a tapered surface 216.

The first inside diameter 221 may be configured to slidably receive the inner shaft 210. The second inside diameter 222 may be less than or equal to the first inside diameter 221. The second inside diameter 222 may be configured to slidably receive the first shaft diameter 213. The first inside diameter 221 and the second shaft diameter 214 may be configured so that the inner shaft 210 and outer shaft 220 are coaxial. Additionally, the second inside diameter 222 and the first shaft diameter 213 may be configured so that the inner shaft 210 and outer shaft 220 are coaxial. The gap between the first shaft diameter 213 and the first inside diameter 221 may be configured to facilitate cleaning and/or flushing through the aperture 219.

The grip portion 223 may be configured to be received within the collet 250. The grip portion 223 may be configured facilitate rotation of the outer shaft 220 in order to threadably engage the first threaded portion 224 of the outer shaft 220 with the inside thread 194 of the surgical screw 190. The grip portion 223 may be configured as a hexagon, square, octagon, or other non-circular geometry.

The second retaining groove 226 may be configured to receive a locking mechanism 182 of the array 180 to retain the array 180 on the outer shaft 220. The third retaining groove 227 may be configured to receive a dowel pin or other retention feature that engages a location feature 245 of the sleeve 240. The first outside diameter 228 may be smaller than the inside diameter 181 of the array 180. The first outside diameter 228 may be smaller than the second outside diameter 229. The first outside diameter 228 may be configured to be received within the array 180. The second outside diameter 229 may be smaller than the inside diameter 241 of the sleeve 240. The second outside diameter 229 may be configured to be received within the sleeve 240.

The second threaded portion 218 may be configured to threadably engage the inside thread 255 of the collet 250. The tapered surface 216 may include one or more slots 217. The one or more slots 217 may facilitate compression of the tapered surface 216 on the second shaft diameter 214 of the inner shaft 210.

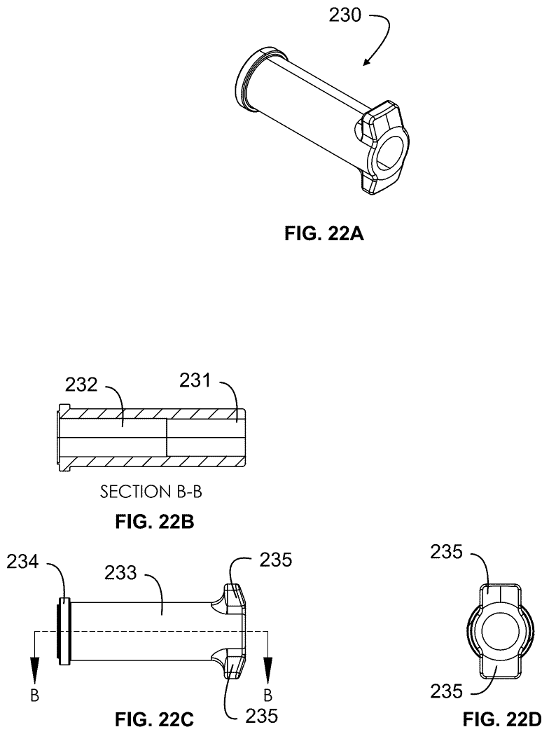

FIG. 22A is a perspective view of a tip 230 of the driver 200 according to an embodiment. FIG. 22B is a section view of the tip 230, FIG. 22C is a front view of the tip 230, and FIG. 22D is a side view of the tip 230. The tip 230 may be configured to engage the inner shaft 210 so that the outer shaft 220 is captive on the inner shaft 210. The tip 230 may be configured to engage the surgical screw 190 between a first arm 192 and a second arm 193. The tip 230 may further be configured to provide a counter-torque to the surgical screw 190 as the first threaded portion 224 of the outer shaft 220 engages the inside thread 194 of the surgical screw 190.

The tip 230 may include a first inside diameter 231, a second inside diameter 232, an outside diameter 233, a flange 234, and one or more wings 235. The first inside diameter 231 may be larger than the drive tip 211 of the inner shaft 210. The first inside diameter 231 may be configured to slidably receive the drive tip 211. The second inside diameter 232 may be configured to securely engage the threaded portion 212 of the inner shaft 210. The second inside diameter 232 may be configured as an internal thread configured to threadably engage the threaded portion 212. The outside diameter 233 may be configured to be received between the first arm 192 and second arm 193 of the surgical screw 190 and may be smaller than a minor diameter of the first threaded portion 224 of the outer shaft 220.

The flange 234 may be equal to or smaller than a minor diameter of the first threaded portion 224 and larger than the second inside diameter 222 of the outer shaft 220 thereby preventing the outer shaft 220 from being removed from the inner shaft 210. Additionally, the flange 234 may not interfere with the first threaded portion 224 of the outer shaft 220 from engaging the inside thread 194 of the surgical screw 190. The one or more wings 235 may be smaller than the distance between the first arm 192 and the second arm 193 of the surgical screw 190.

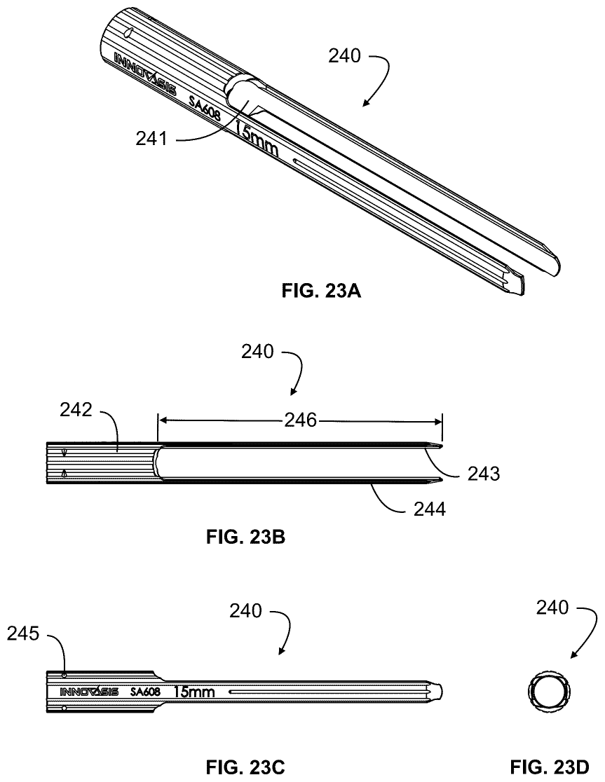

FIG. 23A is a perspective view of a sleeve 240 of the driver 200 according to an embodiment. FIG. 23B is a top view of the sleeve 240, FIG. 23C is a front view of the sleeve 240, and FIG. 23D is a side view of the sleeve 240. The sleeve 240 may be configured to be received within a surgical instrument holding device. The sleeve 240 may be configured to be received within an end effector of a robotic assisted surgical device.

The sleeve 240 may include an outside diameter 242 and an inside diameter 241. The outside diameter 242 may be greater than or equal to a screw head diameter 196 of the surgical screw 190. The inside diameter 241 may be less than the screw head diameter 196.

The sleeve 240 may be sized to be slidably received within an aperture of an engagement mechanism of a surgical instrument holding device. The sleeve 240 may be configured to facilitate engagement of a surgical system 2000 with a surgical instrument holding device, thereby facilitating placement of a surgical screw 190, in which the screw head diameter 196 is smaller than the aperture of an engagement mechanism of the surgical instrument holding device, using the surgical instrument holding device.

The outside diameter 242 may be configured to provide a bearing surface shaped to contact an end effector. The sleeve 240 may be configured to rotate independently of the outer shaft 220 to facilitate threaded engagement and threaded disengagement of the surgical screw 190. More specifically, the outside diameter 242 may be configured to provide a bearing surface for a Ø15 mm end effector.

The sleeve 240 may further include an inside diameter 241, a third arm 243, a fourth arm 244, a location feature 245 and an arm length 246. The inside diameter 241 may be configured to slidably receive the second outside diameter 229 of the outer shaft 220. The third arm 243 may be configured to be received between the first arm 192 and the second arm 193 of the surgical screw 190. The fourth arm 244 may be configured to be received between the first arm 192 and the second arm 193 of the surgical screw 190. The arm length 246 may be less than the arm length 197 of the surgical screw 190. Alternatively, the arm length 246 may be greater than or equal to the arm length 197 of the surgical screw 190.

The location feature 245 may be configured to allow rotation of the sleeve 240 about the outer shaft 220 while preventing translation of the sleeve 240 along an axis. The location feature 245 may include one or more dowel pins configured to be received within a third retaining groove 227 of the outer shaft 220. Alternatively, the location feature 245 may include an o-ring, a snap ring, a retaining ring, a set screw, or other type of retention mechanism know in the art.

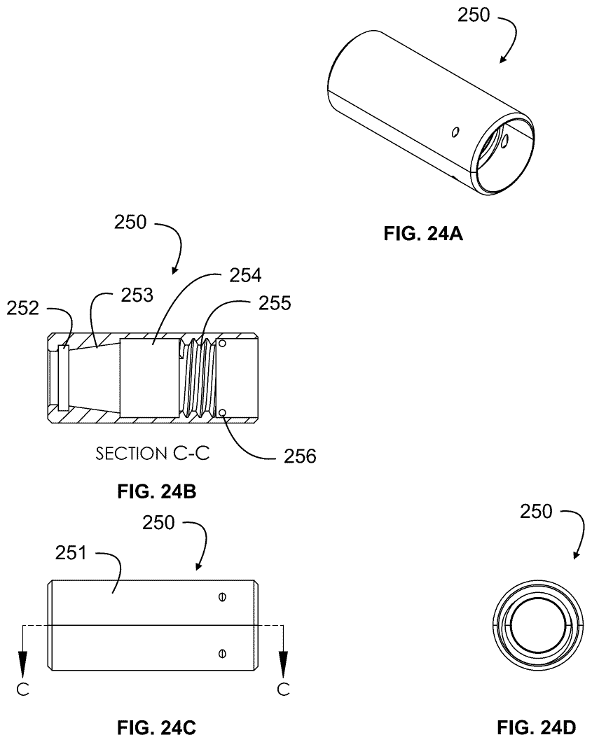

FIG. 24A is a perspective view of a collet 250 of the driver 200 according to an embodiment. FIG. 24B is a section view of the collet 250, FIG. 24C is a front view of the collet 250, and FIG. 24D is a side view of the collet 250. The collet 250 may be configured to receive the outer shaft 220. The collet 250 may include a locking taper 253 configured to engage the tapered surface 216 of the outer shaft 220. The locking taper 253 may be configured to compress the tapered surface 216 onto the second shaft diameter 214 of the inner shaft 210 thereby preventing rotation of the outer shaft 220 with respect to the inner shaft 210.

The collet 250 may also include an outside diameter 251, an inside groove 252, an inside diameter 254, an inside thread 255, and a location feature 256. The outside diameter 251 may be smaller than the inside diameter 181 of the array 180. Additionally, the outside diameter 251 may include a roughened surface configured to improve the grip of a user during rotational movement of the collet 250. The roughened surface may include a knurled pattern on the outside diameter 251. Alternatively, the roughened surface may include a radial pattern of flat surfaces. Alternatively, the collet 250 may have a non-circular cross-sectional profile.

The inside groove 252 may be configured to receive an o-ring. The inside thread 255 may be configured to threadably receive a second threaded portion 218 of the outer shaft 220. The inside diameter 254 may be configured to receive a proximal end of the outer shaft 220. The inside diameter 254 may be larger than the second threaded portion 218 of the outer shaft 220.

The location feature 256 may be configured to make the collet 250 captive on the outer shaft 220. The location feature 256 may be configured to allow rotation of the sleeve 240 about the outer shaft 220 while limiting translation of the sleeve 240 along an axis. The location feature 256 may include one or more dowel pins configured to be received distal to the second threaded portion 218 of the outer shaft 220. Alternatively, the location feature 256 may include an o-ring, a snap ring, a retaining ring, a set screw, or other type of retention mechanism know in the art.

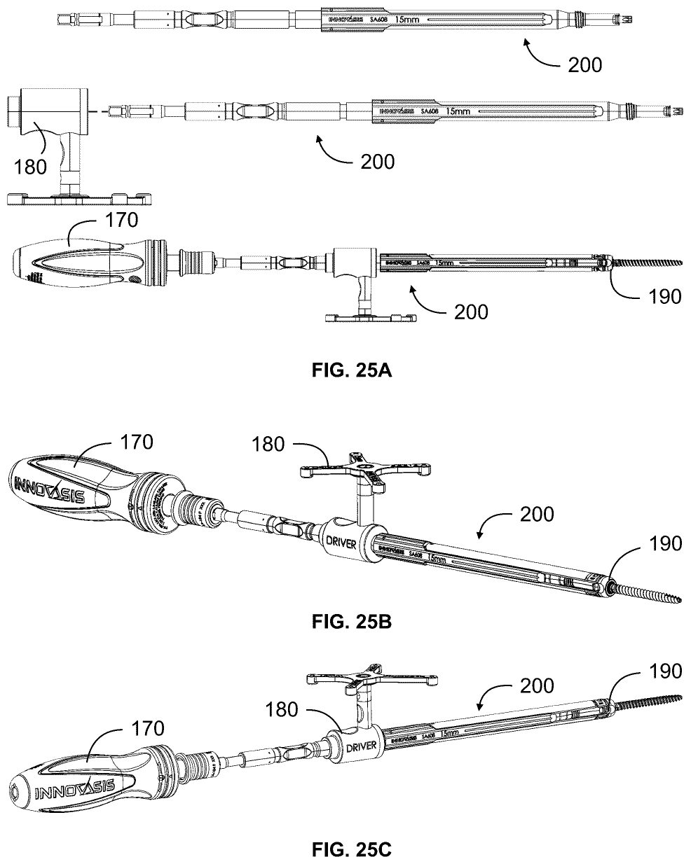

FIG. 25A illustrates steps for assembling a surgical system 2000 according to an embodiment. The method for assembling a surgical system 2000 may include the following steps.
1. Slide the array 180 over the proximal end of the driver 200.
2. Secure the array 180 to the driver 200.
3. Engage the surgical screw 190 on the distal end of the driver 200.
4. Rotate the outer shaft 220 to threadably engage the surgical screw 190.
5. Once the surgical screw 190 is fully seated onto the driver 200, the collet 250 can be turned clockwise until resistance is met, thus inhibiting rotation of the outer shaft 220 and preventing the driver 200 from unintentionally unthreading from the driver 200 during placement of the surgical screw 190.
6. Couple the handle 170 to the driver 200.
7. Use the surgical system 2000 for placement of the surgical screw 190 into a bone.
8. After surgical screw placement:
9. Turn the collet 250 counter-clockwise to allow rotation of the outer shaft 220.
10 Unthread the driver 200 from the surgical screw 190
11. Remove the handle 170
12. Unsecure & remove the array 180.

FIG. 25B is a perspective view of a surgical system 2000, including a driver 200, a handle 170, a surgical screw 190 and an array 180, according to an embodiment. FIG. 25C is a perspective view of the surgical system 2000. The array 180 may be configured to removably receive the outer shaft 220 of the driver 200. The handle 170 may be configured to removably couple with the inner shaft 210 of the driver 200.

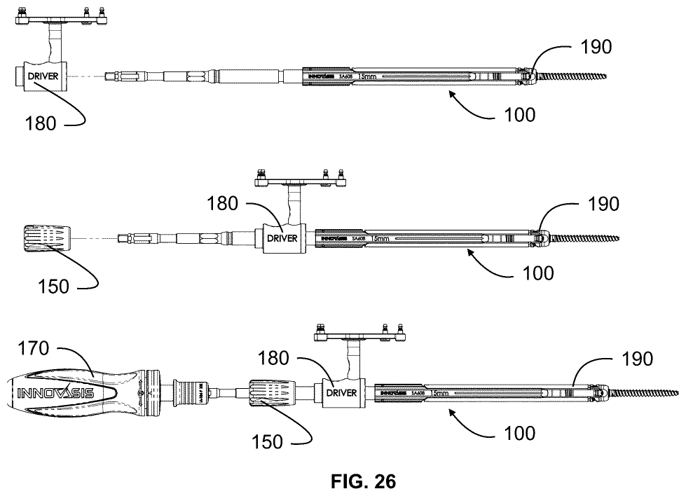

FIG. 26 illustrates steps for assembling a surgical system 1000 according to an embodiment. The method for assembling a surgical system 1000 may include the following steps.
1. Slide the array 180 over the proximal end of the driver 100.
2. Secure the array 180 to the driver 200.
3. Slide the thumb wheel 150 on the driver 100 and engage the drive portion 153 of the thumb wheel 150 with the drive portion 123 of the outer shaft 120.
4. Engage the surgical screw 190 on the distal end of the driver 100.
5. Rotate the thumb wheel 150 to threadably engage the surgical screw 190.
6. Couple the handle 170 to the driver 100.
7. Use the surgical system 1000 for placement of the surgical screw 190 into a bone.
8. After surgical screw placement:
9. Unthread the driver 100 from the surgical screw 190
10. Remove the handle 170
11. Remove the thumb wheel 150
12. Unsecure & remove the array 180.

Those of skill in the art will recognize that this is only one of many potential methods that may be used for an assembling a surgical system for driving a surgical screw. Further, the method may be employed to deploy other surgical fasteners besides those which are specifically disclosed herein.

Reference throughout this specification to “an embodiment” or “the embodiment” means that a particular feature, structure or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, Figure, or description thereof for the purpose of streamlining the disclosure. This method of disclosure, however, is not to be interpreted as reflecting an intention that any claim require more features than those expressly recited in that claim. Rather, as the following claims reflect, inventive aspects lie in a combination of fewer than all features of any single foregoing disclosed embodiment. Thus, the claims following this Detailed Description are hereby expressly incorporated into this Detailed Description, with each claim standing on its own as a separate embodiment. This disclosure includes all permutations of the independent claims with their dependent claims.

The phrases “generally parallel” and “generally perpendicular” refer to structures that are within 30° parallelism or perpendicularity relative to each other, respectively. Recitation in the claims of the term “first” with respect to a feature or element does not necessarily imply the existence of a second or additional such feature or element. Elements recited in means-plus-function format are intended to be construed in accordance with 35 U.S.C. § 112 Para. 6. It will be apparent to those having skill in the art that changes may be made to the details of the above-described embodiments without departing from the underlying principles of the disclosure.

While specific embodiments and applications of the present disclosure have been illustrated and described, it is to be understood that the disclosure is not limited to the precise configuration and components disclosed herein. Various modifications, changes, and variations which will be apparent to those skilled in the art may be made in the arrangement, operation, and details of the methods and systems of the present disclosure without departing from its spirit and scope.

1. A surgical system configured for placement of a surgical screw into a bone, the surgical system comprising:
the surgical screw comprising:
a screw head comprising a screw head diameter, a first arm, a second arm, and an inside thread; and
a screw body coupled with the screw head and comprising a drive feature and a threaded portion configured to engage the bone; and
a driver comprising:
a distal end configured to engage the surgical screw and a proximal end opposite the distal end;
an outer shaft comprising a threaded portion configured to engage the inside thread;
an inner shaft comprising a drive tip configured to engage the drive feature; and
a sleeve configured to be received within an end effector of a robotic assisted surgical device, the sleeve comprising an outside diameter and an inside diameter;
wherein:
the outside diameter is greater than or equal to the screw head diameter;
the inside diameter is less than the screw head diameter; and
the sleeve is further configured to receive the first arm and the second arm.

2. The surgical system of claim 1, wherein the outside diameter is configured to provide a bearing surface shaped to contact the end effector.

3. The surgical system of claim 1, further comprising a thumb wheel configured to be removably receivable on the proximal end of the driver and engageable with the outer shaft so that rotation of the thumb wheel results in rotation of the outer shaft, wherein the outer shaft is configured to rotate independently of the sleeve to facilitate threaded engagement and threaded disengagement of the surgical screw.

4. The surgical system of claim 3, further comprising an array configured to facilitate relative motion tracking of the surgical system, wherein the array is configured to be received on the proximal end of the driver and secured to the outer shaft.

5. The surgical system of claim 1, further comprising a handle configured to be removably receivable on the proximal end of the driver and engageable with the inner shaft so that rotation of the handle results in rotation of the inner shaft.

6. The surgical system of claim 1, wherein the sleeve further comprises a third arm and a fourth arm, wherein the third arm and the fourth arm are configured to be received between the first arm and the second arm.

7. The surgical system of claim 1, wherein the inner shaft is configured to rotate independently of the outer shaft and the sleeve.

8. A surgical system configured for placement of a surgical screw into a bone, the surgical system comprising:
a driver comprising:
a distal end configured to engage the surgical screw and a proximal end opposite the distal end;
a sleeve configured to be received within an end effector of a robotic assisted surgical device, the sleeve comprising an outside diameter and an inside diameter;
an outer shaft configured to be received in the sleeve and comprising a threaded portion configured to threadably engage the surgical screw; and
an inner shaft configured to be received in the outer shaft and rotatable independently of the outer shaft; and
an array configured to facilitate relative motion tracking of the surgical system;
wherein:
the outside diameter is configured to provide a bearing surface shaped to contact the end effector; and
the array is configured to be received on the proximal end of the driver and secured to the outer shaft.

9. The surgical system of claim 8, further comprising the surgical screw comprising:
a screw head comprising a screw head diameter; and
a screw body coupled with the screw head and configured to engage the bone;
wherein:
the outside diameter is greater than or equal to the screw head diameter; and
the inside diameter is less than the screw head diameter.

10. The surgical system of claim 9, wherein the screw head further comprises a first arm and a second arm and the sleeve is further configured to receive the first arm and the second arm.

11. The surgical system of claim 8, further comprising a thumb wheel configured to be removably receivable on the proximal end of the driver and engageable with the outer shaft so that rotation of the thumb wheel results in rotation of the outer shaft, wherein the outer shaft is configured to rotate independently of the sleeve to facilitate threaded engagement and threaded disengagement of the surgical screw.

12. The surgical system of claim 8, further comprising a handle configured to be removably receivable on the proximal end of the driver and engageable with the inner shaft so that rotation of the handle results in rotation of the inner shaft.

13. The surgical system of claim 8, wherein the sleeve further comprises a third arm and a fourth arm and the sleeve further comprises a first arm and a second arm, wherein the third arm and the fourth arm are configured to be received between the first arm and the second arm.

14. The surgical system of claim 8, further comprising the surgical screw comprising a screw head comprising a second arm length and a screw body coupled to the screw head, wherein the sleeve further comprises a first arm length, wherein the first arm length is less than or equal to the second arm length.

15. A surgical system configured for placement of a surgical screw into a bone, the surgical system comprising:
the surgical screw comprising:
a screw head comprising a first arm, a second arm, and an inside thread; and
a screw body coupled with the screw head and comprising a drive feature and a threaded portion configured to engage the bone; and
a driver comprising:
a distal end configured to engage the surgical screw and a proximal end opposite the distal end;
an outer shaft comprising a threaded portion configured to engage the inside thread;
an inner shaft comprising a drive tip configured to engage the drive feature; and
a sleeve configured to be received within an end effector of a robotic assisted surgical device, the sleeve comprising a third arm and a fourth arm;
wherein the third arm and the fourth arm are configured to be received between the first arm and the second arm.

16. The surgical system of claim 15, the screw head further comprises a screw head diameter, wherein:
the sleeve further comprises an outside diameter and an inside diameter;
the outside diameter is greater than or equal to the screw head diameter;
the inside diameter is less than the screw head diameter; and
the sleeve is further configured to receive the first arm and the second arm.

17. The surgical system of claim 15, further comprising a thumb wheel configured to be removably receivable on the proximal end of the driver and engageable with the outer shaft so that rotation of the thumb wheel results in rotation of the outer shaft, wherein the outer shaft is configured to rotate independently of the sleeve to facilitate threaded engagement and threaded disengagement of the surgical screw.

18. The surgical system of claim 15, further comprising a handle configured to be removably receivable on the proximal end of the driver and engageable with the inner shaft so that rotation of the handle results in rotation of the inner shaft.

19. The surgical system of claim 15, the sleeve further comprising an outside diameter, wherein the outside diameter is configured to provide a bearing surface shaped to contact the end effector.

20. The surgical system of claim 15, further comprising an array configured to facilitate relative motion tracking of the surgical system, wherein the array is configured to be received on the proximal end of the driver and secured to the outer shaft.
